(19) United States
(12) Patent Application Publication
(10) Pub. No.: US 2006/0293293 A1
(43) Pub. Date: Dec. 28, 2006

(54) Salmeterol and ciclesonide combination

(75) Inventors: Helgert Muller, Radolfzell (DE); Tushar P. Shah, Flemington, NJ (US)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/556,863

(22) PCT Filed: May 19, 2004
(86) PCT No.: PCT/EP04/50846

§ 371 Date: Nov. 15, 2005

Related U.S. Application Data
(60) Provisional application No. 60/472,466, filed on May 22, 2003.

(51) Int. Cl. A61K 31/58; A61K 31/4745; A61K 31/137

(52) U.S. Cl. 514/171; 514/291; 514/649

(57) ABSTRACT

The invention relates to pharmaceutical compositions containing combinations of salmeterol and ciclesonide and the use of such pharmaceutical compositions in medicine, in particular, the prophylaxis and treatment of respiratory disease.

### FIELD OF THE INVENTION

This invention relates to pharmaceutical compositions containing combinations of salmeterol and ciclesonide and the use of such pharmaceutical compositions in medicine, in particular in the prophylaxis and treatment of respiratory disease.

### BACKGROUND

Salmeterol, which is the compound 4-hydroxy-a¹-[[[6-(4phenylbutoxy)hexyl]amino]-methyl]-1,3-benzenedimethanol, is disclosed in GB 2140800. Salmeterol is known to have stimulant activity at B₂-adrenoreceptors and can be used in the therapy or prophylaxis of conditions susceptible to amelloration by a compound possessing selective stimulant action at B₂-adrenoreceptors, particularly of diseases associated with reversible airway obstruction such as asthma and chronic bronchitis.

GB 247680 discloses pregna-1,4-diene3,20-dione-16-17-acetal-21 esters and their use in the treatment of inflammatory conditions. The compounds have the general structure: 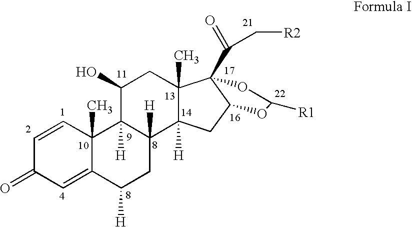
wherein R1 is 2-propyl, 1-butyl, 2-butyl, cyclohexyl or phenyl; and R2 is acetyl or isobutanoyl. Ciclesonide is the INN for a compound of formula I in which R1 is cyclohexyl and R2 is isobutanoyl with the chemical name [11β,16α(R)]-16,17[(Cyclohexylmethylen)bis(oxy)]-11-hydroxy-21-(2-methyl-1-oxoprop-oxy)pregna-1,4dien-3,20-dion.

This compound has undergone evaluation as an antiasthmatic and pharmacokinetic studies show that it will be useful in an inhaler formulation. Ciclesonide is only moderately absorbed after oral administration and has low systemic activity. Concentration of the drug in the lungs is high and metabolism by liver oxidases is very high, giving the drug a low plasma half-life. Systemic activity of ciclesonide is three times lower than that of budesonide, but anti-inflammatory activity is higher for the former.

DE 19541689 is related to the combined use of ciclesonide with a B₂-sympathomimetic, for the treatment of disorders of the respiratory tract.

EP 0 416 951 B1 is related to a combination of salmeterol and fluticasone. It said that the invention is based on the concept of the novel combination therapy which has markedly greater efficiency and duration of bronchodilatory action than previously known combinations and which permits the establishment of a twice daily (bis in diem—b.i.d.) dosing regimen with consequent substantial benefits in, for example, the treatment of asthma, particularly nocturnal asthma.

WO03/013547 is related to a composition comprising salmeterol, budesonide and a carrier for inhalation.

### SUMMARY OF THE INVENTION

It has now been surprisingly found that by combined administration of ciclesonide and salmeterol a significant unexpected therapeutic benefit, particularly a synergistic therapeutic benefit, in the treatment of inflammatory or obstructive airways diseases can be obtained. In particular, it has been found that compositions containing ciclesonide and salmeterol induce an anti-inflammatory activity which is significantly greater than that induced by ciclesonide and salmeterol alone and that the amount of ciclesonide needed for a given anti-inflammatory effect may be significantly reduced when used in admixture with salmeterol, thereby reducing the risk of undesirable side effects from the repeated exposure to the steroid involved in the treatment of inflammatory of obstructive airways diseases. Furthermore, using the compositions of the invention, pharmaceutical compositions, which have a rapid onset and a long duration of action may be prepared. In particular the combination therapy according to the inventions permits the establishment of a twice daily, in particular once daily dosing regimen with consequent substantial benefits in, for example the treatment of obstructive or inflammatory airways diseases (e.g. higher patient compliance, less side effects).

Thus in one aspect the present invention relates to a pharmaceutical composition comprising ciclesonide, a pharmaceutically acceptable salt, solvent or physiologically functional derivative thereof and salmeterol, a pharmaceutically acceptable salt, solvent or physiologically functional derivative thereof and a pharmaceutically acceptable carrier and/or one or more excipients, and optionally one or more other therapeutic ingredients.

Ciclesonide (hereinafter also referred to as active ingredient) is the INN for a compound with the chemical name [11β,16α(R)]-16,17-[(Cyclohexylmethylen)bis(oxy)]-11-hydroxy-21(2-methyl-1-oxoprop-oxy)pregna-1,4-dien3,20dion. Ciclesonide and its preparation are disclosed in DE 4129535. Ciclesonide as used herein also includes, pharmaceutically acceptable salts of ciclesonide, solvates of ciclesonide, physiologically functional derivatives of ciclesonide or solvates thereof. By the term “physiologically functional derivative” is meant a chemical derivative of ciclesonide having the same physiological function as ciclesonide, for example, by being convertible In the body thereto or by being an active metabolite of ciclesonide. Physiological functional derivatives of ciclesonide which may be mentioned in connection with the invention are for example the 21-hydroxy derivative of ciclesonide with the chemical name 16α,17-(22R,S)-Cyclohexylmethylendioxy-11β,21-dihydroxypregna-1,4dien-3,20-dion, in particular 16α,17-(22R)-Cyclohexylmethylendioxy-11β,21-dihydroxypregna-1,4-dien3,20-dion. This compound and its preparation are disclosed in WO 9422899.

Salmeterol (hereinafter also referred to as active ingredient) is the compound 4-hydroxy-a′-[[[6-(4-phenylbutoxy)hexyl]amino]-methyl]-1,3-benzenedimethanol, and is disclosed in GB 2140800. Salmeterol as used herein also includes, pharmaceutically acceptable salts of salmeterol, solvates of salmeterol, physiologically functional derivatives of salmeterol or solvates thereto As would be appreciated by the skilled person, salmeterol includes an asymmetric centre. The present invention includes both (S) and (R) enantiomers of salmeterol either in substantially pure form or admixed in any proportions. The enantiomers of salmeterol have been described previously, for example, in EP0422889 and WO99/13887. Particularly preferred in this connection is the optically pure (S)-enantiomer i.e. (S)-salmeterol. By the term “physiologically functional derivative” is meant a chemical derivative of salmeterol having the same physiological function as the free compound, for example, by being convertible in the body thereto. According to the present invention, examples of physiologically functional derivatives include esters. Suitable salts according to the invention include those formed with both organic and inorganic acids. Pharmaceutically acceptable acid addition salts include but are not limited to those formed from hydrochloric, hydrobromic, sulphuric, citric, tartaric, phosphoric, lactic, pyruvic, acetic, trifluoroacetic succinic, oxalic, fumaric, maleic, oxaloacetic, methanesulphonic, ethanesulphonic, p-toluenesulphonic, benzenesulphonic, isethionic, and naphthalenecarboxylic, such as 1-hydroxy-2-naphthalenecarboxylic acids (hydroxynaphtoate; also referred to by the INN xinafoate).

It will be appreciated that the compounds of the combination may be administered simultaneously, either in the same pharmaceutical formulation (hereinafter also referred to as fixed combination) or in different pharmaceutical formulations (hereinafter also referred to as free combination) or sequentially in any order. If there is sequential administration, the delay in administering the second compound should not be such as to lose the beneficial therapeutic effect of the combination.

As mentioned above, both ciclesonide and salmeterol and their pharmaceutically acceptable salts, solvates, and physiologically functional derivatives have been described for use in the treatment of respiratory diseases. Therefore, formulations of ciclesonide and salmeterol pharmaceutically acceptable salts, solvates, and physiologically functional derivatives have use in the prophylaxis and treatment of clinical conditions for which a selective B₂-adrenoreceptor agonist and/or a glucocorticosteroid is indicated. Such conditions include diseases associated with reversible airways obstruction such as asthma, nocturnal asthma, exercise-induced asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis).

Accordingly, the present invention provides a method for the prophylaxis or treatment of a clinical condition in a mammal, such as a human, for which a selective B₂-adrenoreceptor agonist and/or glucocorticosteroid is indicated, which comprises administration of a therapeutically effective amount of a pharmaceutical formulation comprising salmeterol or a pharmaceutical acceptable salt, solvate, or physiologically functional derivative thereof and ciclesonide or a pharmaceutical acceptable salt, solvate, or physiologically functional derivative thereof, and a pharmaceutical acceptable carrier and/or one or more excipients. In a preferred aspect, there is provided such a method, which comprises administration of a therapeutically effective amount of a pharmaceutical formulation comprising salmeterol xinafoate and ciclesonide, and a pharmaceutical acceptable carrier and/or one or more excipients. In particular, the present invention provides such a method for the prophylaxis or treatment of a disease associated with reversible airways obstruction such as asthma, chronic obstructive pulmonary disease (COPD), respiratory tract infection or upper respiratory tract disease.

The amount of salmeterol and ciclesonide, or a pharmaceutical acceptable salt, solvate or physiologically functional derivative thereof which is required to achieve a therapeutic effect will, of course, vary with the particular compound, the route of administration, the subject under treatment, and the particular disorder or disease being treated. As a monotherapy, salmeterol xinafoate is generally administered to adult humans by aerosol inhalation at a dose of 50 μg or 100 μg twice daily. As a monotherapy, ciclesonide is generally administered to adult humans by inhalation at a daily dose of from 0.05 to 1.6 mg, preferably 0.05 to 1 mg, which can be administered in one or several doses. It is preferred in connection with the present invention to have a twice daily and particularly preferred to have a once daily dosing regimen.

Suitably, the pharmaceutical formulations which are suitable for inhalation according to the invention comprise the active ingredients in amounts such that in case of administration by inhalation from inhalers each actuation provides a therapeutically effective dose, for example, a dose of salmeterol of 10 μg to 150 μg, preferably 50 μg and a dose of ciclesonide of 10 μg to 800 μg, 25 μg to 500 μg, 40 μg to 400 μpreferably 50 μg to 200 μg, more preferably, 50 μg to 100 μg. It is particularly preferred that each actuation provides a dose therapeutically effective for a twice daily dosing regiment or more particularly preferred for a once daily dosing regimen. The pharmaceutical formulations according to the invention may further include other therapeutic agents for example anticholinergics such as ipatropium and tiotropium, pharmaceutically acceptable salts salts or solvents thereof. Examples, which may be mentioned are ipatropium bromide and tiotropium bromide and solvates thereof.

Suitably, the pharmaceutical formulations which are suitable for inhalation according to the invention provide therapeutically effective doses that permit the establishment of a twice daily (bis in diem—b.i.d) dosing regimen and in particular a once daily dosing regimen.

The formulations include those suitable for oral, parenteral (including subcutaneous, intradermal, intra-muscular, intravenous and intraaarticular, intranasal, inhalation (including fine particle dusts or mists which may be generated by means of various types of metered dose pressurised aerosols, nebulisers or insufflators), rectal and topical (including dermal, buccal, sublingual and intraocular administration) although the most suitable route may depend upon for example the condition and disorder of the recipient. The formulations may conveniently be presented in unit dosage form and may be prepared by any of the methods well known in the art of pharmacy. All methods include the step of bringing the active ingredients into association with the carrier, which constitutes one or more accessory ingredients/excipients. In general the formulations are prepared by uniformly and intimately bringing into association the active ingredients with liquid carriers or finely divided solid carriers or both and then, if necessary, shaping the product into the desired formulation. Formulations for inhalation include powder compositions, which will preferably contain lactose, and spray compositions which may be formulated, for example, as aqueous solutions or suspensions or as aerosols delivered from pressurised packs, with the use of a suitable propellant, e. g. 1,1,1,2-terafluorethane, 1,1,1,2,3,3,3-heptafluoropropane, carbon dioxide or other suitable gas. A class of propellants, which are believed to have minimal ozone-depleting effects in comparison to conventional chlorofluorocarbons comprise hydrofluorocarbons and a number of medicinal aerosol formulations using such propellant systems are disclosed in, for example, EP 0372777, W091/0401 1, W091/11173, W091/11495, W091/14422, W093/11743, and EP-0553298. These applications are all concerned with the preparation of pressurised aerosols for the administration of medicaments and seek to overcome problems associated with the use of this new class of propellants, in particular the problems of stability associated with the pharmaceutical formulations prepared. The applications propose, for example, the addition of one or more of excipients such as polar cosolvents or wetting agent (e.g. alcohols such as ethanol), alkanes, dimethyl ether, surfactants (including fluorinated and non-fluorinated surfactants, carboxylic acids such as oleic acid, polyethoxylates etc.) or bulking agents such as a sugar (see for example WO02/30394) and vehicles such as cromoglicic acid and/or nedocromil which are contained at concentrations, which are not therapeutically and prophylactically active (see WO00/07567). For suspension aerosols, the active ingredients should be micronised so as to permit inhalation of substantially all of the active ingredients into the lungs upon administration of the aerosol formulation, thus the active ingredients will have a particle size of less than 100 microns, desirably less than 20 microns, and preferably in the range 1 to 10 microns, for example, 1 to 5 microns.

However, despite the various approaches used in formulating drugs for use in aerosol inhalation, there are still many serious difficulties and uncertainties often encountered in attempting to develop a physically and chemically stable CFC-free formulation that reliably delivers an accurate dose of drug having the proper particle size range. In particular, there is a need for a CFC-free medicinal aerosol product containing ciclesonide in combination with salmeterol that is chemically and physically stable and that is suitable for delivery to the respiratory system of a patient.

WO 98/52542 is related to pharmaceutical compositions comprising a therapeutically effective amount of ciclesonide or a related compound and a hydrofluorocarbon propellant, preferably selected from 1,1,1,2-tetrafluoroethane, 1,1,1,2,3,3,-heptafluoropropane and a mixture thereof, and cosolvent, preferably ethanol, in an amount effective to solubilize ciclesonide and optionally a surfactant.

In one aspect the invention relates to a pharmaceutical aerosol composition comprising salmeterol in particulate form and ciclesonide in dissolved form in the carrier. The invention thus relates to a pharmaceutical aerosol composition comprising particles of salmeterol in a therapeutically effective amount and a therapeutically effective amount of ciclesonide and a hydrofluorocarbon propellant, preferably selected from 1,1,1,2-tetrafluoroethane, 1,1,1,2,3,3,-heptafluoropropane and a mixture thereof, and cosolvent, preferably ethanol, in an amount effective to solubilize ciclesonide and optionally a surfactant.

Ciclesonide is generally present in the formulation at a concentration of from 1 to 8 mg/ml, preferably 1 to 5 mg/ml. Such formulation generally comprises ethanol in an amount effective to solubilize the ciclesonide but not salmeterol. The propellant preferably includes a hydrofluoroalkane, in particular Propellant 134a, Propellant 227 or a mixture thereof, generally at about 50:50 w/w. More preferably the propellant consists of Propellant 134a. The formulations may contain surfactant such as oleic acid, but may be also free of surfactant The formulations are preferably free of other excipients.

The formulations may be prepared by preparing a drug concentrate of one or both of the active ingredients with ethanol and adding this concentrate to the pre-chilled propellant in a batching vessel. Preferably a solution of the ciclesonide in the cosolvent is added to the prechilled propellant in a batching vessel and then subsequently salmeterol is added to form a suspension. The resulting formulation is filled into vials. Alternatively the formulations may be prepared by adding the required quantity of active ingredients into an aerosol vial, crimping a valve on the vial and introducing a pre-mixed blend of propellant and ethanol through the valve. The vial is placed in an ultrasonic bath to ensure solubilisation of ciclesonide.

In another embodiment of the invention there is provided a pharmaceutical aerosol composition comprising salmeterol in a therapeutically effective amount and a therapeutically effective amount of ciclesonide and a hydrofluorocarbon propellant, preferably selected from 1,1,1,2-tetrafluoroethane, 1,1,1,2,3,3,3-heptafluoropropane and a mixture thereof, and cosolvent, preferably ethanol, in an amount effective to solubilize ciclesonide and the salmeterol and optionally a surfactant.

Ciclesonide is generally present in the formulation at a concentration of from 1 to 8 mg/ml, preferably 1 to 5 mg/ml. Such formulation generally comprises from 3 to 25% preferably 5 to 25% more preferably 5 to 20%, most preferably 7 to 12% by weight ethanol. The propellant preferably includes a hydrofluoroalkane, in particular Propellant 134a, Propellant 227 or a mixture thereof, generally at about 50:50 w/w. More preferably the propellant consists of Propellant 134a. The formulations may contain surfactant such as oleic acid, but may be also free of surfactant. The formulations are preferably free of other excipients.

The formulations may be prepared by preparing a drug concentrate of both of the active ingredients in ethanol and adding this concentrate to the pre-chilled propellant in a batching vessel. The resulting formulation is filled into vials. Alternatively, the formulations may be prepared by adding the required quantity of active ingredients into an aerosol vial, crimping a valve on the vial and introducing a premixed blend of propellant and ethanol through the valve. The vial is placed in an ultrasonic bath to ensure solubilisation of active ingredients.

In another embodiment preferred compositions for aerosol delivery contain both active ingredients in particulate form, and 1,1,1,2-tetrafluoroethane, 1,1,1,2,3,3,3-heptafluoropropane or mixtures thereof as propellant. Such formulation generally comprises from 0.01 to 5% (w/w relative to the total weight of the formulation) of polar cosolvent, in particular ethanol. In a preferred embodiment no or less than 3% w/w of polar cosolvent, in particular ethanol is contained. Especially preferred compositions for aerosol delivery consist of particulate active ingredients, and 1,1,1,2-tetrafluoroethane, 1,1,1,2,3,3,3-heptafluorpropane or mixtures thereof as propellant and optionally a surfactant (preferably oleic acid).

The formulations may be prepared by adding the required quantity of active ingredients into an aerosol vial, crimping a valve on the vial and introducing propellant or optionally a pre-mixed blend of propellant and optionally the cosolvent and surfactant through the valve.

Canisters generally comprise a container capable of withstanding the vapour pressure of the propellant, such as plastic or plastic-coated glass bottle or preferably a metal can, for example an aluminium can which may optionally be anodised, lacquer-coated and/or plastic-coated, which container is closed with a metering valve. It may be preferred that canisters be coated with a fluorocarbon polymer as described in WO 96/32150, for example, a co-polymer of polyethersulphone (PES) and polytetrafluoroethylene (PTFE). Another polymer for coating that may be contemplated is FEP (fluorinated ethylene propylene).

The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in W092/1190 and valves containing EPDM rubber as described in W095/02650 are especially suitable. Suitable valves are commercially available from manufacturers well known in the aerosol industry, for example, from Valois, France (eg. DF10, DF30, DF60), Bespak pic, UK (eg. BK300, BK356, BK357) and 3M-Neotechnic Ltd, UK (eg. Spraymiser).

Valve seals, especially the gasket seal and also the seals around the metering chamber, will preferably be manufactured of a materials which is inert to and resists extraction into the contents of the formulation, especially when the contents include ethanol.

Valve materials, especially the material of manufacture of the metering chamber, will preferably be manufactured of a material which is inert to and resists distortion by contents of the formulation, especially when the contents include ethanol. Particularly suitable materials for use in manufacture of the metering chamber include polyesters eg polybutyleneterephthalate (PBT) and acetals, especially PBT.

Materials of manufacture of the metering chamber and/or the valve stem may desirably be fluorinated, partially fluorinated or impregnated with fluorine containing substances in order to resist drug deposition.

Valves, which are entirely or substantially composed of metal components (eg Spraymiser, 3M-Neotechnic), are especially preferred for use according to the invention.

Intranasal sprays may be formulated with aqueous or non-aqueous vehicles with or without the addition of agents such as thickening agents, buffer salts or acid or alkali to adjust the pH, isotonicty adjusting agents preservatives or anti-oxidants. Suitable aqueous formulations for application to mucosa are for example disclosed in WO01/28562 and WO01/28563.

In another embodiment of the invention the pharmaceutical formulation comprising the ciclesonide in combination with salmeterol is a dry powder, i.e. salmeterol and ciclesonide are present in a dry powder comprising finely divided ciclesonide and salmeterol optionally together with a finely divided pharmaceutically acceptable carrier, which is preferably present and may be one or more materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose, particularly in the form of the monohydrate. The dry powder may be in capsules of gelatins or plastic, or in blisters, for use in a dry powder inhalation device, preferably in dosage units of the mixture of ciclesonide and salmeterol together with the carrier in amounts to bring the total weight of powder in each capsule to from 5 mg to 50 mg. Alternatively the dry powder may be contained in a reservoir of a multi-dose dry powder inhalation device. Capsules and cartridges or for example gelatin, or blisters of for example laminated aluminium foil, for use in an inhaler or insulator may be formulated containing a powder mix of the active ingredients and a suitable powder base such as lactose or starch, preferably lactose. In this aspect, the active ingredients are suitably micronised so as to permit inhalation of substantially all of the active ingredients into the lungs upon administration of the dry powder formulation, thus the active ingredients will have a particle size of less than 100 μm, desirably less than 20 μm, and preferably in the range 1 to 10 μm. The solid carrier, where present, generally has a maximum particle diameter of 300 μm, preferably 200 μm, and conveniently has a mean particle diameter of 40 to 100 μm, preferably 50 to 75 μm. The particle size of the active ingredients and that of a solid carrier where present in dry powder compositions, can be reduced to the desired level by conventional methods, for example by grinding in an air-jet mill, ball mill or vibrator mill, microprecipitation, spray drying, lyophilisation or recrystallisation from supercritical media.

Where the inhalable form of the composition of the invention is the finely divided particulate form, the inhalation device may be, for example a dry powder inhalation device adapted to deliver dry powder from a capsule or blister containing a dosage unit of the dry powder or a multi-dose dry powder inhalation device. Such dry powder inhalation devices are known in the art Examples which may be mentioned are Cyclohaler®, Diskhaler®, Rotadisk®, Turbohaler® or the dry powder inhalation devices disclosed EP 0 505 321, EP 407028, EP 650410, EP 691865 or EP 725725 (Ultrahaler®).

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product. Suitable technologies for this type of administration are known in the art. As an example the Mystic® technology is to be mentioned (see for example U.S. Pat. No. 6,397,838, U.S. Pat. No. 6,454,193 and U.S. Pat. No. 6,302,331).

Preferred unit dosage formulations are those containing a pharmaceutical effective dose, as hereinbefore recited, or an appropriate fraction thereof, of the active ingredient. Thus, in the case of formulations designed for delivery by metered dose pressurised aerosols, one actuation of the aerosol may deliver half of the therapeutical effective amount such that two actuations are necessary to deliver the therapeutically effective dose.

It should be understood that in addition to the ingredients particularly mentioned above, the formulations of this invention may include other agents conventional in the art having regard to the type of formulation in question. Furthermore, the claimed formulations include bioequivalents as defined by the US Food and Drugs Agency.

The invention will now be illustrated by the following examples without restricting it.

### EXAMPLE 1

### Metered Dose Inhaler

227 g 1,1,1,2,3,3,3-heptafluorpropane (TG 227) are liquefied by cooling to a temperatur of approximately −50° C. A solution of 1.7 g ciclesonide and 10 mg oleic acid in 20 g ethanol (absolute) is added. Subsequently 1.22 g micronised salmeterol xinafoate are added and the suspension formed is homogenized intensively. TG 227 is added to the suspension to a total weight of the composition of 1000 g, while cooling and stirring. The chilled suspension is filled in aluminium cans and a metering valve (25 μl) is crimped into place. Per actuation 25 μl of the suspension corresponding to 50 μg ciclesonide and 36.3 μg salmeterol xinafoate (corresponding to 25 μg salmeterol) are released.

### EXAMPLE 2

### Metered Dose Inhaler

4.52 g ciclesonide and 0.82 g salmeterol xinafoate are added to a vessel containing 400 g ethanol and the mixture is stirred to give a solution. 1595 g 1,1,1,2-tetrafluorethane (TG 134a) are weight into a pressure vessel. The ethanolic solution containing the active ingredients is subsequently added to the propellant while stirring. The pressurized solution is then filled into aluminum cans with metering valves (75 μl) using standard pressure filling techniques. Per actuation 75 μl of the solution corresponding to 200 μciclesonide and 36.3 μg salmeterol xinafoate (corresponding to 25 μg salmeterol) are released.

### EXAMPLE 3

### Powder Inhaler (Mono Dose System Based on Inhalation Capsule)

800 mg micronised ciclesonide, 290 mg salmeterol xinafoate micronised and 28.9 g lactose monhydrate (Ph. Eur. 4) are mixed in a turbula mixer in two steps. The blend is screened (0.71 mm sieve) and transferred to the container of a planetary mixer. After adding with additional 70.0 g lactose monhydrate and mixing, 25 mg of the blend are filled into capsules of size 3, which can be administered with a powder inhaler. One capsule contains 200 μg ciclesonid and 72.5 μg salmeterol xinafoate (corresponding 50 μg salmeterol).

### EXAMPLE 4

### Powder Inhaler (Multi Dose System)

1000 g lactose monohydrate (Ph. Eur. 4) is screened by a sieve-mill. 3.625 g salmeterol xinafoate micronised (screened; 0.5 mm mesh) and 146.4 g of deagglomerated lactose monohydrate are blended in a turbula mixer. 195 g of deagglomerated lactose monohydrate are filled in a high shear mixer and 5.0 g of ciclesonide micronised (screened, 0.5 mm sieve) are added to form a blend. The salmeterol lactose preblend is screened (0.5 mm sieve), added to the container of a high shear mixer and mixed with the ciclesonide lactose blend. Subsequently 650 g of deagglomerated lactose monohydrate are added and mixed. 1.5 g of the blend are filled In the reservoir of a multi dose powder inhaler. After fully assembling, the powder inhaler is wrapped into a protective foil to achieve moisture protection. Such powder inhaler will contain 60 single doses (20 mg powder) each containing 100 μg ciclesonide and 72.5 μg salmeterol xinafoate (corresponding to 50 μg salmeterol).

### EXAMPLE 5

### Powder Inhaler (Multi Dose System)

1.93 g micronised salmerol xinafoate and 18.1 g lactose monohydrate (Ph. Eur. 4) are screened (0.5 mm sieve) and mixed in a turbula mixer. The blend obtained is screened (0.5 mm sieve) and together with 10.67 g micronised ciclesonide (screened; mesh 0.5 mm) and 196.3 g lactose monohydrate (Ph. Eur. 4) filled in a steel batching vessel and blended in a turbula mixer. 1.2 g of the blend thus obtained is filled in the powder reservoir of a powder inhaler. After fully assembling the powder inhaler is wrapped in a protective foil to achieve protection from moisture. Such powder inhaler may contain at least 120 single doses (7.5 mg powder) each having 400 μg ciclesonide and 72.5 μg salmeterol xinafoate (corresponding to 50 μg salmeterol).

### EXAMPLE 6

### Solution for Nebulisation

Appropriate amounts of ciclesonide and salmeterol are dissolved in a ethanol containg 10% water. 2.5 ml of the solution are filled in containers which can be used in Respimat® device.

Although the invention has been described in terms of preferred formulations and ingredients, it will be understood that these are not intended to be limiting. To the contrary, those skilled in the art will understand that various optional ingredients may be included, such as flavouring agents, preservatives, additional active ingredients, and the like, while still embodying the present invention.

1. A pharmaceutical composition comprising ciclesonide or a pharmaceutically acceptable salt or solvate thereof or a physiologically functional derivative of ciclesonide or solvate thereof and salmeterol or a pharmaceutically acceptable salt or solvate thereof or a physiologically functional derivative of salmeterol or solvate thereof, and a pharmaceutically acceptable carrier and/or one or more excipients, and optionally one or more other therapeutic ingredients.

2. The composition according to claim 1, wherein ciclesonide and salmeterol are contained in the same pharmaceutical formulation.

3. The composition according to claim 1, wherein the salmeterol is salmeterol xinafoate.

4. The composition according to claim 1, wherein the salmeterol is optically pure (S)-salmeterol.

5. The composition according to claim 1, comprising ciclesonide and salmeterol in an amount and ratio to be effective for a twice or once daily treatment of a clinical condition in a mammal for which a selective β₂-adrenoreceptor agonist and/or glucocorticosteroid is indicated.

6. The composition according to claim 1, additionally containing tiotropium bromide or solvates thereof.

7. The pharmaceutical composition according to claim 1, which is suitable for administration by inhalation.

8. A pharmaceutical aerosol composition comprising particles of salmeterol in a therapeutically effective amount and a therapeutically effective amount of ciclesonide and a hydrofluorocarbon propellant, and a cosolvent in an amount effective to solubilize ciclesonide and optionally a surfactant.

9. The pharmaceutical composition according to claim 8, wherein the cosolvent is ethanol.

10. A pharmaceutical aerosol composition comprising particles of salmeterol in a therapeutically effective amount and particles of ciclesonide in a therapeutically effective amount and a hydrofluorocarbon propellant, and 0.01 to 5% w/w based upon propellant of polar cosolvent and optionally a surfactant.

11. The pharmaceutical composition according to claim 7, which is a dry powder and the carrier is lactose monohydrate.

12. A method for the treatment of a clinical condition in a mammal for which a selective β₂-adrenoreceptor agonist and/or glucocorticosteroid is indicated, which comprises administering a therapeutically effective amount of a pharmaceutical formulation comprising salmeterol or a pharmaceutical acceptable salt or solvate thereof, or a physiologically functional derivative of salmeterol or a solvate thereof, and ciclesonide or a pharmaceutical acceptable salt or solvate thereof, or a physiologically functional derivative of ciclesonide or a solvate thereof, and a pharmaceutical acceptable carrier and/or one or more excipients.

13. The method according to claim 12, wherein the clinical condition is selected from the group consisting of asthma, nocturnal asthma, exercise-induced asthma, chronic obstructive pulmonary diseases (COPD), chronic and wheezy bronchitis, emphysema, respiratory tract infection, upper respiratory tract disease, rhinitis, allergic rhinitis and seasonal rhinitis.

14. The method according to claim 12, which comprises a twice daily dosage regimen.

15. The method according to claim 12, which comprises a once daily dosage regimen.

16. The method according to claim 12, which comprises administering a combination of the ciclesonide and salmeterol in the same administration form by inhalation from an inhaler and wherein each actuation provides a dose therapeutically effective for a twice daily dosing regimen or for a once daily dosing regimen.

17. A dry powder inhalation product comprising a pharmaceutical composition according to claim 7.

18. A pharmaceutical aerosol product comprising an aerosol vial equipped with a metering valve and containing an aerosol formulation suitable for oral or nasal inhalation comprising a pharmaceutical aerosol composition according to claim 8.

19. A pharmaceutical aerosol product comprising an aerosol vial equipped with a metering valve and containing an aerosol formulation suitable for oral or nasal inhalation comprising a pharmaceutical aerosol composition according to claim 9.

20. A pharmaceutical aerosol product comprising an aerosol vial equipped with a metering valve and containing an aerosol formulation suitable for oral or nasal inhalation comprising a pharmaceutical aerosol composition according to claim 10.

21. The pharmaceutical aerosol composition according to claim 8, wherein the hydrofluorocarbon propellant is selected from the group consisting of 1,1,1,2-tetrafluoroethane, 1,1,1,2,3,3,3-heptafluoropropane and mixtures thereof.

22. The pharmaceutical aerosol composition according to claim 10, wherein the hydrofluorocarbon propellant is selected from the group consisting of 1,1,1,2-tetrafluoroethane, 1,1,1,2,3,3,3-heptafluoropropane and mixtures thereof.
